(19) United States
(12) Patent Application Publication
(10) Pub. No.: US 2023/0157607 A1
(43) Pub. Date: May 25, 2023

(54) IN-EAR ELECTROENCEPHALOGRAPHY DEVICE

(71) Applicant: BIOSERENITY, Paris (FR)

(72) Inventor: Philippe GUERMONPREZ, Paris (FR)

(73) Assignee: BIOSERENITY, Paris (FR)

(21) Appl. No.: 17/922,844

(22) PCT Filed: May 6, 2021
(86) PCT No.: PCT/EP2021/061991

§ 371 Date: Nov. 2, 2022

(30) Foreign Application Priority Data
May 6, 2020 (EP) 20305446.5

(51) Int. Cl. A61B 5/27; A61B 5/00; A61B 5/265; A61B 5/369

(52) U.S. Cl. CPC A61B 5/27; A61B 5/6804; A61B 5/265; A61B 5/6817; A61B 5/369; A61B 2562/0215; A61B 2562/125

(57) ABSTRACT

An in-ear device including a cloth, which defines a concave surface, at least one electrode in or on the cloth, and a conductive track connected to the at least one electrode. Also, a method of monitoring neurological or physiological diseases or disorders with the in-ear device and a method of collecting electroencephalography data with the in-ear device. Further, a process for manufacturing the in-ear device.

### FIELD OF INVENTION

The present invention pertains to the field of electroencephalogram (EEG) devices. In particular, the invention relates to an in-ear electroencephalography device. Said device is particularly configured to enable the diagnosis, monitoring or treatment of neurological or physiological diseases or disorders requiring electrical brain activity measurements.

### BACKGROUND OF INVENTION

The EEG is a sample of electrical brain activity. A minimum of 20 minutes of artefact-free recording (including activation procedures) is necessary to assess baseline brain electrical activity. Longer recordings improve the chance of recording an abnormality and of demonstrating their variability. Moreover, the medical monitoring of persons undergoing their usual activities is useful in many cases. Thus, monitoring means must not disrupt or disturb the subject's life, for example in the case of the elderly, during sleep or in other situations.

The present invention aims at providing a device that measures electrical brain activity for a long period of time, for example for more than 20 minutes, while being compact, comfortable, ergonomic and easy to use. Such a device allows an easier examination and a more reliable diagnosis and/or monitoring.

### SUMMARY

The present invention relates to an in-ear device comprising a cloth defining a concave surface, at least one electrode in or on said cloth, and at least one conductive track connected to said at least one electrode. In a preferred embodiment, said cloth has a shape of a sleeve which has dimension adapted to fit into at least a portion of the auditory canal of an ear of a patient. Advantageously, the hollow shape of the cloth allows the in-ear device of the invention to be highly multifunctional. Indeed, the hollow shape of the cloth allows to accommodate into the ear of a subject nor only the in-ear device but also any other desired object and/or instruments matching the dimension of the in-ear device and the ear of the subject. Therefore, advantageously the EEG recording may be acquired from the electrodes of the in-ear device for example, while an object is attenuating the sound that reaches the ear (i.e., ear plug) or while an ear speaker diffuses a sound into the ear.

According to one embodiment, said cloth is a knitted textile, a non-woven textile, a woven textile or a combination thereof. Advantageously, the use of a textile cloth improves the comfort of the subject carrying the in-ear device into his/her auditory canal.

Preferably, said cloth is made of polyamide.

According to one embodiment, said at least one electrode is a textile electrode that is woven, knit, stitched, stuck or deposited in or on said cloth.

In one embodiment, said at least one electrode and/or said conductive track is made of silvered polyamide, advantageously allowing an improved comfort for the subject that needs to wear the in-ear device during many hours.

According to one embodiment, said in-ear device comprises at least two electrodes, preferably at least 3 electrodes, more preferably at least 4 electrodes, even more preferably at least 5 electrodes.

In one embodiment, said electrodes are disposed on either side of a plane that passes through the longitudinal axis of the in-ear device, and faces each other.

In another embodiment, said electrodes are disposed on either side of a plane that passes through the longitudinal axis of the in-ear device, and are shifted from each other's.

In another embodiment, said electrodes are disposed on either side of a plane perpendicular to the longitudinal axis of the in-ear device.

According to one embodiment, said in-ear device further comprises an ear plug that is at least partially surrounded by the cloth. In this embodiment, the ear plug is configured to be fitted inside the hollow space of the sleeve shape cloth and therefore has dimension adapted to fit the cloth and, as consequence, the ear canal of a subject.

In one embodiment, said in-ear device further comprises an instrument such as a microphone or an earphone, said instrument is at least partially surrounded by the cloth. In this embodiment, the ear plug is configured to be fitted inside the hollow of the cloth and therefore has dimension adapted to fit the cloth and, as consequence, the ear canal of a subject.

The present invention also relates to a method of monitoring neurological or physiological diseases or disorders, comprising the setting up of an in-ear device according to the present invention in an ear of a subject and the measurement of an electrical activity.

The present invention also relates to a method of collecting electroencephalography data, comprising the setting up of an in-ear device according to the present invention in an ear of a subject and the measurement of an electrical activity.

The present invention further relates to a process for manufacturing an in-ear device comprising the following steps:
i. knitting a cloth with an insulating yarn and an electrode with a conductive yarn in order to obtain a cloth defining a concave surface, preferably in the form of a sleeve or a sheath, comprising said electrode in or on said cloth;
ii. connecting the at least one electrode with a conductive track; and
iii. optionally slipping the cloth obtained at step i) on an ear plug.

### DEFINITIONS

In the present invention, the following terms have the following meanings:
“Abnormal brain activity” refers to brain electrical activity that is present in brain disorders and that is different from physiological activity, such as for example interictal epileptiform discharges and electrographic seizures (“seizure” refers to a transient occurrence of signs and/or symptoms due to abnormal, excessive and synchronous neuronal activity in the brain), known to be characteristic for epilepsy.
“About” preceding a figure means plus or less 10% of the value of said figure.
“Cloth” refers to an article to be worn by a subject. A cloth may comprise a textile material, having a certain cohesion.
“Concave surface” refers to a depressed surface, whose shape is hollow. According to one embodiment, said concave surface is a sleeve, preferably a sleeve that is closed at one end.
“Conductive” refers to the ability to allow the flow of charge (electrical current) in one or more directions. In this disclosure, a material enabling electrical connections between elements of a circuit without degrading significantly EEG measure is conductive. For instance, the electrical conductivity σ of a conductive material is greater than 100 milli-siemens per centimetre (mS/cm). “Non-conductive” and “Insulated” refers to the non-ability or the weak ability to allow the flow of charge (electrical current) in one or more directions. In this disclosure, a material providing electrical isolation between elements of a circuit to ensure reliable EEG measure is non-conductive. For instance, the electrical conductivity σ of a non-conductive or insulating material is less than 1 micro-siemens per centimetre (μS/cm).
“Electroencephalogram” or “EEG” refers to the record of the electrical activity of the brain of a subject, preferably made by electrodes.
“Electrode” refers to a conductor used to establish electrical contact with a non-metallic part of a circuit, preferably a subject body. “Textile electrodes” refers to an electrode whose structure is a textile, in particular a textile knitted from a conductive yarn or woven from a conductive yarn or non-woven from conductive fibres.
“Length of electrode” refers to the measurement of the electrode edge(s) that is perpendicular to the longitudinal axis of the in-ear device. The “Width of electrode” refers to the measurement of the electrode edge(s) that is parallel to the longitudinal axis of the in-ear device. Therefore, the length of the electrode may be smaller than the width of the same electrode.
“Signal processing means” denotes at least one microprocessor, at least one integrated circuit, at least one electronic board and at least one microcontroller. This signal processing means may comprise an assembly including an onboard computer and external computing means such as a mobile device and remote servers.
“Sleep disorder” denotes a medical trouble whose origin can be physiological, environmental or behavioural (related to an individual's sleep patterns).
“Sleeve” is used in the present application as a general term that can refer to a cylindric form, either open at both ends or closed at least at one end, or that can refer to a conical shape if both ends do not have the same diameter, said conical shape being either open at both ends or closed at least at one end. If the sleeve is closed at one end, it can also be designated by the term sheath.
“Subject” refers to a mammal, preferably a human In the sense of the present invention, a subject may be a patient, i.e. a person receiving medical attention, undergoing or having underwent a medical treatment, or monitored for the development of a disease or for the follow-up during or after treatment.
“Textile” refers to a material comprising textile fibres. A textile may be obtained by assembling yarns, fibers and/or filaments by any suitable method such as, for example, weaving, knitting, compressing, sticking, needling, or any suitable method known by the person skilled in the art. “Woven textile” refers to a textile that is obtained by weaving yarns and that consists in a textile structure with crossed horizontal and vertical yarns for example manufactured on a loom. “Non-woven textile” refers to a material made from fibres bonded together by chemical, mechanical, heat or solvent treatment, for example by needling, compressing, sticking, or by any other suitable method.
“Track” refers to a conductive element. According to one embodiment, the conductive track may consist of one or more conductive yarns, fibers and/or filaments. Said conductive yarns, fibers and/or filaments are made of a conductive material, or covered with a conductive surface. Preferably, said conductive yarns, fibers and/or filaments are made of polyamide covered with silver. According to another embodiment, the conductive track may also consist of a conductive ink or a conductive paint on a substrate, the conductive ink or paint is charged with an electrically conductive material having flexible properties, allowing this conductive ink or paint to be deposited on flexible surfaces such as a cloth.

### DETAILED DESCRIPTION

The present invention relates to an in-ear device comprising a cloth defining a concave surface, at least one electrode in or on said cloth, and at least one conductive track connected to said at least one electrode.

According to one embodiment, said concave surface is a sleeve. Said sleeve can be either open at both ends, open at one end and closed at the other end, or closed at both ends.

Preferably, said sleeve is closed at least at one end, more preferably said sleeve is open at one end and closed at the other end (see FIG. 1 and FIG. 2).

The in-ear device and in particular the cloth is dimensioned so as to be insertable into the ear canal of a subject. According to one embodiment, the shape of said cloth is like the shape of an ear plug (see FIG. 2): a cone whose beginning diameter h is between 1 and 20 mm, preferably between 5 and 15 mm, more preferably between 5 and 10 mm, even more preferably between 5 and 8 mm, even more preferably about 8 mm or about 5 mm; whose end diameter H is between 5 and 20 mm, preferably between 8 and 13 mm, more preferably between 10 and 13 mm, even more preferably about 13 mm or about 10.5 mm, even more preferably about 11 mm or about 12 mm; and whose length L is between 15 and 35 mm, preferably between 20 and 30 mm, more preferably between 23 and 27 mm, even more preferably about 25 mm or about 23 mm.

According to a preferred embodiment, the shape of said cloth is a cone whose beginning diameter h is about 8 mm, whose end diameter H is about 10 mm, and whose length L is about 25 mm.

In one feature, said cloth is at least slightly elastic, in order to advantageously conform to the shape of the ear canal. The elasticity of said cloth allows the manufacture of only one size of said cloth as if it is sufficiently elastic so that it can fit various adult ear canals.

The material of the cloth is compatible with skin contact and is non-conductive. According to one embodiment, the cloth is made of textile. Preferably, said cloth is a knitted textile, a non-woven textile, a woven textile or a combination thereof. The use of a knitted textile and/or a woven textile for manufacturing the cloth advantageously provides the desired elasticity to said cloth. More preferably, said cloth is made of polyamide. Even more preferably, said cloth is a knitted textile made of polyamide, such as the polyamide 6,6.

Advantageously, said cloth is seamless, for example a seamless knitted cloth. The absence of seams improves comfort as it allows the surface of the cloth to be smooth. In addition, a smooth surface improves the quality of contact between electrode and skin of the subject, and hence improves the quality of electrical signal measured.

The in-ear device comprises at least one electrode in or on the cloth that defines a concave surface. Said at least one electrode is(are) able to measure electrical brain activity. The in-ear electrode should be comfortable in order to permit the wearer to sleep. The electrode should if possible, not be elastic in order to have a constant size. The material of the in-ear electrode should be compatible for skin contact.

According to one embodiment, said at least one electrode is a textile electrode that is woven, knit, stitched, sticked or deposited in or on said cloth. According to a preferred embodiment, said at least one electrode is made of silvered polyamide. According to another preferred embodiment, said at least one electrode is made by two-yarns knitting, one yarn being of polyamide 6,6 and one yarn being of silvered polyamide.

Preferably, the cloth of the in-ear device is a knitted textile and the at least one textile electrode(s) is/are knitted in said knitted cloth. In other words, the cloth and the at least one electrode represent only one textile, said textile comprising areas made of conductive yarns and areas made of non-conductive yarns, delimiting conductive areas that are the at least one electrode(s) and insulating areas that are the cloth. In this embodiment, the in-ear device is a seamless knitting, which is more comfortable and provide with better electrical signal because it has a smooth surface. More preferably, the conductive yarns are silvered polyamide yarns and/or the non-conductive yarns are polyamide 6,6 yarns.

Alternatively, the conductive areas are made by two-yarns knitting, one yarn being of polyamide 6,6 and one yarn being of silvered polyamide, and the insulating areas are made by knitting polyamide 6,6 yarns.

Moreover, concerning polyamide 6,6 and silver polyamide, the biocompatibility of the polyamide 6,6 and of silver is well-known, they are very safe materials.

According to another embodiment, said at least one electrode is made of an elastic ink that comprises silver or silver chloride, and is preferably deposited on said cloth.

According to one embodiment, said in-ear device comprises one electrode (see FIG. 3a). Preferably, when said in-ear device comprises one electrode, two in-ear devices are used (one in each ear of a subject) and/or a gel electrode stick is placed behind the ear in which the in-ear device is present (FIG. 4) or behind the other ear or anywhere on the body preferably away from an electrophysiological signal source (brain, heart, muscles). Indeed, a second electrode is necessary to establish a potential difference: the electrical activity measured at the second electrode (reference electrode) location is subtracted from the electrical activity measured at the target location (active electrode).

According to alternative embodiment, the in-ear device comprises 2, 3, 4, 5 or 6 electrodes (see FIG. 3b), FIG. 3c) and FIG. 3d). Advantageously, the more electrodes are present in or on said cloth, the more the signal resolution is improved, that is that the system is able to map signals from smaller areas of the brain. Indeed, high resolution means that the system is able to map signals from small areas of the brain. Advantageously, the resolution that could be obtained with multiple electrodes is of the order of a centimetre. The use of non-conductive yarns of the cloth allows to place multiple electrodes in one in-ear device.

The at least one electrode of said in-ear device can be positioned in or on said cloth in any way suitable for measurements of electrical signal.

In one embodiment, the position of the electrodes is invariable by rotation along the axis of the cloth. If there is rotational symmetry along the axis of the cloth, there is no need to orient the direction of the cloth in the ear of the subject, and therefore the use of the in-ear device is easier.

In one embodiment, said electrodes are disposed on either side of a plane that passes through the longitudinal axis of the in-ear device, and facing each other. In another embodiment, said electrodes are disposed on either side of a plane that passes through the longitudinal axis of the in-ear device, and shifted from each other's. In another embodiment, said electrodes are disposed on either side of a plane perpendicular to the longitudinal axis of the in-ear device.

The in-ear electrode should go deep in the ear, as deep as an ear plug (about 23 mm). Preferably, the at least one electrode starts at between 1 and 6 mm from the top of the concave-shaped cloth, preferably at between 2 mm and 5 mm, more preferably at about 4 mm (the deepest part in the ear).

According to one embodiment, the length of the at least one electrode is between 10 and 20 mm, preferably between 12 and 18 mm, more preferably between 15 and 17 mm, even more preferably about 15 mm or about 17 mm; and the width of the at least one electrode is between 1 and 8 mm, preferably between 2 and 6 mm, more preferably between 4 and 5 mm, even more preferably about 4 mm or about 5 mm For example, as shown in FIG. 5, if the in-ear device comprises 2 electrodes that are disposed on either side of a plane perpendicular to the longitudinal axis of the in-ear device and that overlap at their ends, the one that is the closest to the top of the concave-shaped cloth can have a length of about 15 mm and a width of about 5 mm, and the one that is the closest to the base of the concave-shaped cloth can have a length of about 17 mm and a width of about 4 mm.

In one embodiment, the at least one electrode has shape and dimension to form a closed loop around the cloth of the in-ear device.

The shape of the electrodes may be any shape suitable for measuring electrical brain activity, for example square, round, oval, rectangular, diamond shaped etc.

According to one embodiment, said at least one electrode does not require gel for use, preferentially said at least one reference electrode is a dry conductive electrode.

As explained above, the cloth is elastic and has dimension and shape adapted to fit the auditory canal the subject. Notably, such a cloth allows to ensure a contact between the in-ear device and internal walls of the auditory canal. The electrodes of the in-ear device are made of conductive material allowing to record the electric activity from the inner wall of the auditory canal without the need of a gel, such as electrolytic cream. The electrodes of the in-ear device are as well adapted for use with a gel. The electrolytic cream may allow to improve the conductivity between the skin and the electrode, thus improving the electrical signal measured. Advantageously, the textile structure of the cloth absorbs the electrolytic cream and releases said cream once the in-ear device is in place in the ear channel; this ensures that there is enough cream on said electrodes and prevents the cream from spreading out on the cloth between said electrodes, which can produce short circuits.

The in-ear device comprises a cloth defining a concave surface, at least one electrode in or on said cloth, and a conductive track connected to said at least one electrode. Preferably, there is one track per electrode.

According to one embodiment, said conductive track is a textile track, preferably a knitted textile track, more preferably a textile track made by knitting silvered polyamide yarns.

In one embodiment, said track goes from the electrode to the edge of the cloth or further than the edge of the cloth for the electrical connection to an acquisition system of electrical brain activity.

Preferably, the conductive track is positioned inside the cloth of the in-ear device in order to be not in contact with the skin outside the auditory canal.

According to an advantageous feature, the materials of the in-ear device, for example polyamide 6,6 for the cloth and silvered polyamide for the at least one electrode and the at least one conductive track, allows said in-ear device to be washed at least 20 times (in conditions suitable to remove ear wax) and/or is of sufficient low price to be disposable. For example, the in-ear device can be washed in a laundry washing machine.

The in-ear device has a thickness that allows to have a large specific surface area in contact with skin and/or electrolytic cream when said in-ear device is crushed in the ear of a subject, which lowers the contact impedance and improves the EEG signal quality. For example, the thickness of said in-ear device is between 0.1 and 5 mm, preferably between 0.1 and 3 mm, more preferably between 0.1 and 1 mm, even more preferably between 0.1 and 0.5 mm, even more preferably between 0.2 and 0.4 mm, even more preferably about 0.3 mm.

According to one embodiment, the in-ear device according to the present invention, as described above, further configured to cooperate or comprises an ear plug and/or an instrument, the cloth being configured to fit the ear plug and/or the instrument. In one feature, the cloth is removably slipped on the ear plug and/or the instrument. For example, the instrument can be a microphone or an earphone. For example, the ear plug can be a foam ear plug, such as an earplug made of polyurethane. Notably, the ear plug and/or the instrument is inserted in the hollow of the sleeve-shaped cloth so that the ear plug and/or the instrument is at least partially surrounded by the cloth. Moreover, the ear plug and/or the instrument has dimensions adapted to fit the auditory canal when inserted into the hollow of the sleeve-shaped cloth. In one preferred embodiment, the outer shape of the ear plug and/or the instrument fits the inner surface of the cloth so that when the in-ear device is inserted into the auditory canal, the ear plug and/or the instrument pushes the electrodes against the internal wall of the auditory canal.

The in-ear device can also further comprise an ear plug and several instruments, provided that the cloth of said in-ear device can fit them. Alternatively, the in-ear device can also further comprise several instruments and no ear plug, provided that the cloth of said in-ear device can fit these instruments.

Indeed, as the cloth of the in-ear device has a concave surface, its hollow allows to put an ear plug and/or an instrument. When the cloth fit at the same time an ear plug and an instrument such as an earphone or a microphone, the ear plug can have been cut at its top in order to allow space for said instrument.

As mentioned above, the cloth of the in-ear device can be at least slightly elastic; this elasticity of said cloth allows the manufacture of only one size of said cloth as if it is sufficiently elastic it can fit the various adult ear canal and the various ear plug sizes.

According to one embodiment, said in-ear device is connected to an acquisition system configured to acquire the signal measured by the at least one electrode and the connexion is detachable. The acquisition system comprises an electronic circuit.

In one embodiment, said acquisition system further comprises a signal processing means. According to one embodiment, said signal processing means comprises a memory unit, said memory unit comprising neurological markers; and the signal processing means is configured to identify a neurological disorder among the signals measured by the at least one electrode based on the neurological markers.

For example, the memory unit can comprise epileptic markers; the identification of epileptic activity among the signals measured by the at least one electrode can use an epileptic activity recognition algorithm based on a neural network analysis.

According to one embodiment, the signal processing means of the in-ear device is configured to identify or annotate the side-effects of a medical treatment via an electronic data collection, said side-effects resulting in changes to electrical brain activity. This collection may be direct (input of subject's data in a software application) or indirect (collection via a third-party computing system).

According to one embodiment, the signal processing means is configured to identify the subject's sleep cycles based on the various signals acquired by the acquisition system.

According to one embodiment, the in-ear device also comprises or is connected to at least one remote storage means and means for communicating to at least one cloud computing or remote server type remote storage means.

Advantageously, said acquisition system further comprises means for transmitting the electrical signal acquired by the acquisition system to said signal processing means. The transmission means is configured to transmit the electrical signals acquired by the acquisition system to the signal processing means. In one embodiment, the signal processing means is remote from this device and is connected wirelessly. In one embodiment, the signal processing means is remote from this device and is connected in a wired manner.

Preferably, the in-ear device comprises a cloth defining a concave surface, at least one electrode in or on said cloth, a conductive track connected to said at least one electrode, and is connected to an acquisition system, which comprises a transmission means and a signal processing means. In one embodiment, the in-ear device comprises a plurality of acquisition systems, transmission means and signal processing means.

An object of the present invention is also a process for manufacturing an in-ear device comprising the following steps:
i. knitting at least one electrode with a conductive yarn and a cloth with an insulating yarn in order to obtain a cloth defining a concave surface, preferably in the form of a sleeve or a sheath, said at least one electrode being in or on said cloth;
ii. connecting the at least one electrode with a conductive track; and
iii. optionally slipping the cloth obtained at step i) on an ear plug and/or on at least one instrument.

According to one embodiment, the insulating yarn used in step i. is a polyamide 6,6 yarn.

According to one embodiment, the conductive yarn used in step i. is a silvered polyamide yarn.

According to one embodiment, the insulating yarn used in step i. is a polyamide 6,6 yarn and the conductive yarn used in step i. is a silvered polyamide yarn. One advantage of using polyamide for both cloth and electrode is that the cloth and the at least one electrode as a whole is homogeneous and similarly flexible in all points.

According to one embodiment, the conductive track at step ii. is made by knitting silvered polyamide yarn.

In one embodiment, said cloth and said at least one electrode are knitted on an automatic knitting machine which ensures a better manufacturing quality and reproducibility and which lowers the manufacturing cost, allowing to consider a single-use of the cloth comprising the at least one electrode.

The automatic knitting machine may be of the glove knitting type, with a special program for knitting the textile at least one electrode. For example, said knitting machine can be the machine whose reference is SWG041 and which is manufactured by the company SHIMA, with 240 needles and a gauge of 15.

After step i., once the cloth comprising at least one electrode is knitted, some yarns are shaved and the cloth comprising at least one electrode is washed according to an industrial process called “end of production”. This washing process ensures the cleaning of the sizing oils (used to facilitate knitting) and cleans possible handling stains.

The present invention also relates to a method of monitoring neurological or physiological diseases or disorders, comprising the setting up of an in-ear device in an ear of a subject and the measurement of electrical activity.

The present invention further relates to a method of collecting electroencephalography data, comprising the setting up of an in-ear device in an ear of a subject and the measurement of electrical activity.

According to one embodiment, the in-ear device can be used for capturing an EEG of a subject. This EEG is a useful for diagnosing, monitoring and/or treating neurological or physiological diseases or disorders requiring electrical brain activity measurements, such as epilepsy or sleep disorders.

The invention also relates to the use of an in-ear device according to the present invention, for monitoring neurological or physiological diseases or disorders requiring electrical brain activity measurements.

Advantageously, the device can be set up on a subject by the subject himself or by any other person such as health or non-health professional. Normally mucous membranes of the auditory canal are protected by wax but for a better skin electrode contact the patient will be asked to clean the wax of his/her ears. An electrolytic cream can be used on the at least one electrode.

The size, location and texture of the device, that is an in-ear device comprising a cloth, improves discreetness and user comfort, especially over long periods of time. The in-ear device is comfortable in order to permit the wearer to sleep. For example, for a polysomnography examination, the subject has to wear the device for a period between 4 hours and 48 hours.

Advantageously, the integration of textile electrode(s) such as electrode(s) made of silvered polyamide yarns, in or on the fabric or textile of the cloth, allows a better comfort for the subject that needs to wear the in-ear device during many hours.

The present invention enables a reliable analysis of the electrical brain signal measured by detecting the periods during which said signal is disturbed.

The in-ear device maybe used with the 10-20 international system, the 10-10 system or the 10-5 system.

While various embodiments have been described and illustrated, the detailed description is not to be construed as being limited hereto. Various modifications can be made to the embodiments by those skilled in the art without departing from the true spirit and scope of the disclosure as defined by the claims.

### BRIEF DESCRIPTION OF THE DRAWINGS

FIG. 1 is a drawing of an embodiment of an in-ear device.

FIG. 2 is a schema representing an embodiment of a cloth of an in-ear device.

FIG. 3 is divided in four schemas (FIGS. 3a, 3b), 3c) and 3d)) representing some of the possible dispositions of the at least one electrode in or on the cloth of an in-ear device.

FIG. 4 represents four ways to use in-ear devices.

FIG. 5 is two schemas representing dimensions of two electrodes that are perpendicular to the longitudinal axis of an in-ear device.

FIG. 6 is a picture of an embodiment of an in-ear device.

### ILLUSTRATIVE EMBODIMENT OF THE INVENTION

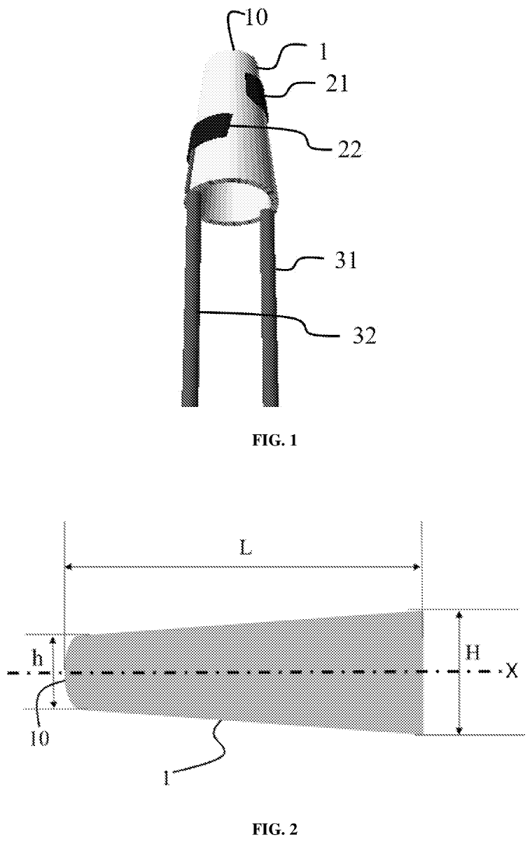

As shown in FIG. 1, in a specific embodiment of an in-ear device according to the present invention, said in-ear device comprises a cloth 1 defining a concave surface, two electrodes (21, 22) in said cloth, and one conductive track (31, 32) connected to each electrode. Said cloth is made of polyamide 6,6 and said electrodes are made of silvered polyamide, the cloth and the electrodes being knitted at once, as a whole, by changing the yarn for delimiting the insulated areas (that is the cloth 1) and the conductive areas (that are the electrodes (21, 22)). Moreover, said cloth has a shape of a sleeve and in particular of a cone that is closed at its top 10 and open at its base.

The cloth 1 has the following dimensions (see FIG. 2): the beginning diameter h (at the top (10)) is about 8 mm, the end diameter H (at the base) is about 13 mm, and the length L is about 25 mm. The longitudinal axis X of the in-ear device is represented in FIG. 2.

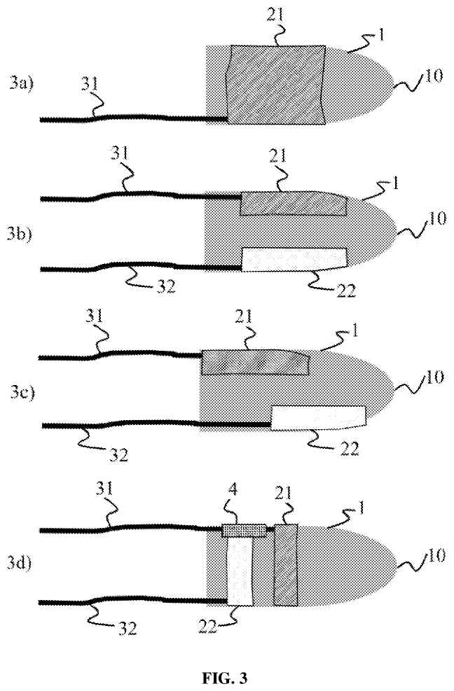

As shown in FIG. 3, the in-ear device can comprise one electrode 21 (FIG. 3a)) or two electrodes (21, 22) (FIGS. 3b), 3c) and 3d)). When at least two electrodes (21, 22) are present, they can be disposed according to several manners in or on the cloth 1: for example, they can be disposed on either side of a plane that passes through the longitudinal axis X of the in-ear device delimited by the cloth, and facing each other's (FIG. 3b)), or they can be disposed on either side of a plane that passes through the longitudinal axis X of the in-ear device, and shifted from each other's (FIG. 3c)), or they can be disposed on either side of a plane perpendicular to the longitudinal axis X of the in-ear device (FIG. 3d)). Each electrode (21, 22) us connected to one conductive track (31, 32).

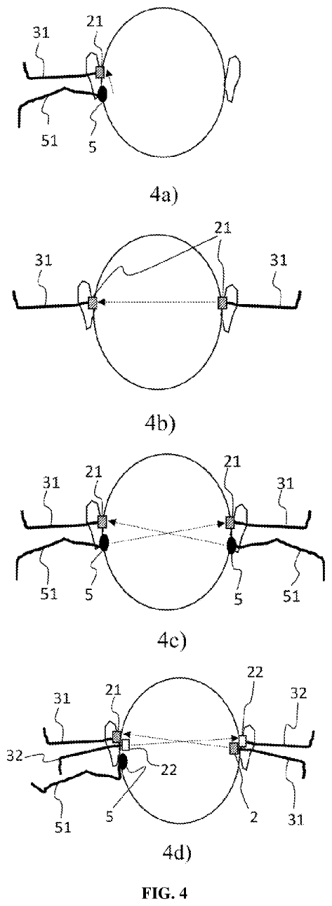

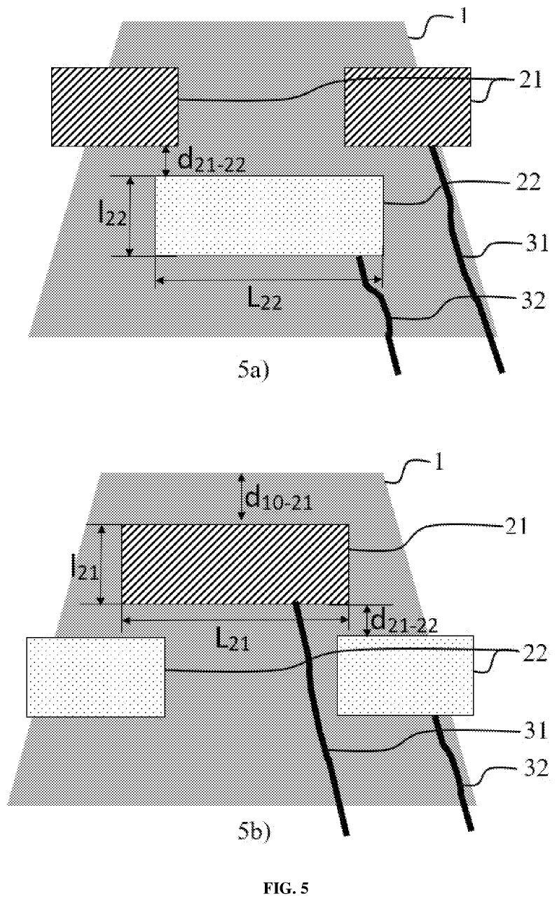

FIG. 4 illustrates various embodiments of uses of in-ear devices. The use of in-ear devices comprising one electrode, includes the use of another in-ear device in the other ear (FIG. 4b) and/or the use of a gel electrode stick placed behind the ear in which the in-ear device is present (FIG. 4a and FIG. 4c). Indeed, a second electrode is necessary to establish a potential difference: the electrical activity measured at the second electrode (reference electrode) location is subtracted from the electrical activity measured at the target location (the active electrode).

FIG. 4a) shows an embodiment of an in-ear device that comprises only one electrode 21 connected to a conductive track 31 and thus its use is associated to the use of a gel electrode stick 5 placed behind the ear in which the in-ear device is present and which is connected to a conductive track 51.

FIG. 4b) shows an embodiment of an in-ear device that comprises only one electrode 21 connected to a conductive track 31 and thus its use in one ear is associated to the use of another in-ear device that comprises only one electrode 21 connected to a conductive track 31 in the other ear.

FIG. 4c) shows an embodiment of the use of an in-ear device that comprises only one electrode 21 connected to a conductive track 31 in each ear, and the associated use of a gel electrode stick 5 placed behind each ear and which is connected to a conductive track 51.

FIG. 4d) shows an embodiment of the use of an in-ear device that comprises two electrodes (21, 22) connected to a conductive track (31, 32) in each ear, and the associated use of a gel electrode stick 5 placed behind one ear and which is connected to a conductive track 51.

Without wishing to be limited by any theory, using devices in both ears allows for further distance between the active electrode(s) and the reference electrode(s), so as to increase the electrical potential difference.

FIG. 5 shows an embodiment of electrodes dimensions and relative distances between them and the top of the in-ear device according to the invention. According to this embodiment, two electrodes (21, 22) are present on the cloth 1 and are disposed on either side of a plane perpendicular to the longitudinal axis X of the in-ear device. Each electrode (21,22) is connected to conductive track (31, 32). FIG. 5a) represents the in-ear device cut lengthwise in one layer and spread out, the axis of the cut passing through the first electrode 21. The second electrode 22 has a length L₂₂ of 17 mm±1 mm and a width l₂₂ of 4 mm±1 mm; the distance d₂₁₋₂₂ between the both electrodes (21, 22) is 3 mm±1 mm FIG. 5b) represents the in-ear device cut lengthwise in one layer and spread out, the axis of the cut passing through second electrode 22. The first electrode 21 has a length L₂₁ of 15 mm±1 mm and a width l₂₁ of 5 mm±1 mm; the distance d₂₁₋₂₂ between the both electrodes (21, 22) is 3 mm±1 mm; the distance d₁₀₋₂₂ between electrode 21 and the top of the cone-shaped cloth 1 is 4 mm±1 mm.

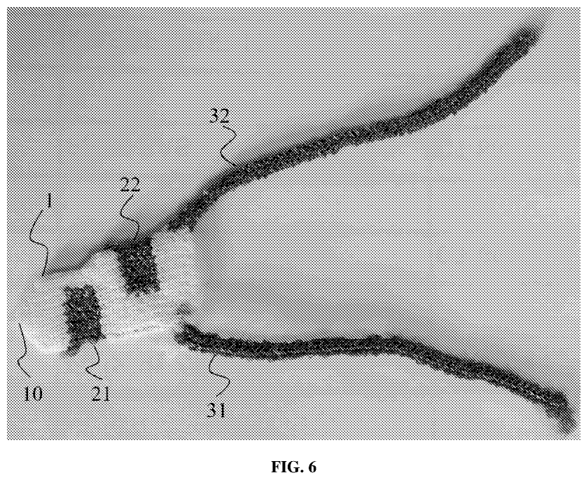

FIG. 6 shows a picture of an in-ear device, that comprises a cloth 1 defining a concave surface, two electrodes (21, 22) in said cloth 1, and two conductive tracks (31, 32), each connected to each electrode. In this embodiment, said cloth is made by knitting polyamide 6,6 yarn and said electrodes are made by two-yarns knitting, one yarn being of polyamide 6,6 and one yarn being of silvered polyamide. The cloth 1 and the electrodes (21, 22) are knitted at once, as a whole, by changing the yarn for delimiting the insulated areas (that is the cloth 1) and the conductive areas (that is the electrodes (21, 22)). Moreover, according to this embodiment, said cloth 1 has a shape of a sleeve and in particular of a cone that is closed at its top 10 and open at its base. Both electrodes (21, 22) are disposed on either side of a plane perpendicular to the longitudinal axis X of the in-ear device, they do not define a loop around the cloth but are facing each other's relative to the longitudinal axis X of the in-ear device.

Said in-ear device illustrated in FIG. 6 has been knitted on a knitting machine whose reference is SWG041 and which is manufactured by the company SHIMA, with 240 needles and a gauge of 15; the duration of the knitting of said in-ear device was less than 5 minutes.

1-14. (canceled)

15. An in-ear device comprising a cloth having a shape of a sleeve, at least one electrode in or on said cloth, and at least one conductive track connected to said at least one electrode.

16. The device according to claim 15, wherein said cloth is a knitted textile, a non-woven textile, a woven textile or a combination thereof.

17. The device according to claim 15, wherein said cloth is made of polyamide.

18. The device according to claim 15, wherein said at least one electrode is a textile electrode that is woven, knit, stitched, stuck or deposited in or on said cloth.

19. The device according to claim 15, wherein said at least one electrode and/or said at least one conductive track is made of silvered polyamide.

20. The device according to claim 15, wherein it comprises at least two electrodes, preferably at least 3 electrodes, more preferably at least 4 electrodes, even more preferably at least 5 electrodes.

21. The device according to claim 20, wherein said electrodes are disposed on either side of a plane that passes through the longitudinal axis of the in-ear device, and facing each other.

22. The device according to claim 20, wherein said electrodes are disposed on either side of a plane that passes through the longitudinal axis of the in-ear device, and shifted from each other.

23. The device according to claim 20, wherein said electrodes are disposed on either side of a plane perpendicular to the longitudinal axis of the in-ear device.

24. The device according to claim 15, further comprising an ear plug at least partially surrounded by the cloth.

25. The device according to claim 15, further comprising an instrument such as a microphone or an earphone, said instrument being at least partially surrounded by the cloth.

26. A method of monitoring neurological or physiological diseases or disorders, comprising the setting up of an in-ear device according to claim 15 in an ear of a subject and the measurement of electrical activity.

27. A method of collecting electroencephalography data, comprising the setting up of an in-ear device according to claim 15 in an ear of a subject and the measurement of electrical activity.

28. A process for manufacturing an in-ear device comprising the following steps:
knitting a cloth with an insulating yarn and an electrode with a conductive yarn in order to obtain a cloth having a shape of a sleeve, preferably in the form of a sleeve or a sheath, comprising said electrode in or on said cloth;
connecting the at least one electrode with a conductive track; and
optionally slipping the cloth obtained at step i) on an ear plug.
